(19) United States
(12) Patent Application Publication
(10) Pub. No.: US 2025/0114628 A1
(43) Pub. Date: Apr. 10, 2025

(54) APPARATUS AND METHOD FOR SELF-ADMINISTERED INTRA-ORAL PHOTOBIOMODULATION

(71) Applicants: Ross Lefkowitz, Philadelphia, PA (US); Brian Pryor, Newark, DE (US); Sean Xiaolu Wang, Wilmington, DE (US); Dan Liu, Hockessin, DE (US); Li Fu, Hockessin, DE (US)

(72) Inventors: Ross Lefkowitz, Philadelphia, PA (US); Brian Pryor, Newark, DE (US); Sean Xiaolu Wang, Wilmington, DE (US); Dan Liu, Hockessin, DE (US); Li Fu, Hockessin, DE (US)

(73) Assignee: SBR Biotechnologies, Inc., Newark, DE (US)

(21) Appl. No.: 18/887,178

(22) Filed: Sep. 17, 2024

Related U.S. Application Data
(60) Provisional application No. 63/542,799, filed on Oct. 6, 2023.

(51) Int. Cl. A61N 5/06

(52) U.S. Cl. CPC A61N 5/06; A61N 2005/0606; A61N 2005/0626; A61N 2005/0632; A61N 2005/0644

(57) ABSTRACT

The photobiomodulation apparatus (and method of use) of the present invention comprises one or more light sources which are integrated into a transparent flexible mouthpiece housing containing a semi rigid support member for providing photobiomodulation therapy to the oral mucosa of a cancer patient suffering from, or at risk of, Oral Mucositis. The light sources can be either internal, or external to the transparent flexible mouthpiece housing, and either lasers, light emitting diodes (LEDs), lamps, or any other types of light sources which produce therapeutic light in a desired wavelength range. The therapeutic light is delivered through the transparent flexible mouthpiece housing to the oral mucosa. The delivered light produces a photochemical and photophysical process e.g. increase the blood flow and circulation. This photochemical and photophysical process helps to reduce the incidence and severity of oral mucositis in cancer patients and helps improve outcomes of cancer therapy.

### CROSS-REFERENCE TO RELATED APPLICATIONS

This application claims inventions which were disclosed in Provisional Patent Application No. 63/542,799, filed Oct. 6, 2023, entitled “APPARATUS AND METHOD FOR SELF-ADMINISTERED INTRA-ORAL PHOTOBIOMODULATION”. The benefit under 35 USC § 119(e) of the above-mentioned United States Provisional Applications is hereby claimed, and the aforementioned application is hereby incorporated herein by reference.

### FIELD OF THE INVENTION

This invention generally relates to a photobiomodulation apparatus (and method of use), and more specifically to a photobiomodulation for treating oral mucositis in cancer patients.

### BACKGROUND OF THE INVENTION

Oral Mucositis is a common side effect of cancer treatment (i.e. chemotherapy and radiation therapy). Oral Mucositis is the severe ulceration or the oral mucosa which is often debilitating, causing patients to stop eating, and can lead to severe complications and morbidity. Photobiomodulation (PBM) therapy (a therapeutic dose of light to the tissue) is considered effective for preventing and treating oral mucositis incidence and severity. Currently, only in-office PBM treatments are available which require patients to travel to an office each day to get this potentially lifesaving treatment. Accordingly, there is a need for a self-administered, home use photobiomodulation therapy apparatus for patients at risk of or suffering from Oral Mucositis.

### SUMMARY OF THE INVENTION

The photobiomodulation apparatus (and method of use) of the present invention comprises one or more light sources which are integrated with a transparent flexible mouthpiece housing for providing photobiomodulation therapy to the oral mucositis of cancer patients suffering from or at risk of Oral Mucositis. The light sources can be lasers, light emitting diodes (LEDs), lamps, or any other types of light sources which produce therapeutic light in a desired wavelength range. The transparent flexible mouthpiece housing contains a semi rigid support member which allows the mouthpiece to maintain form when it is bent by a patient. This form maintaining property is essential to provide effective, quick, and comfortable self-administer treatment. The therapeutic light is delivered through the mouthpiece to key oral mucosa, such as, but not limited to, buccal mucosa (bilateral), tongue (dorsum), side of tongue (bilateral), tongue (ventral)/floor of mouth, hard palate, and soft palate. The delivered light produces a photochemical and photophysical process e.g., increase the blood flow and circulation. This photochemical and photophysical process helps to reduce the incidence and severity of oral mucositis in cancer patients and helps improve outcomes of cancer therapy.

### BRIEF DESCRIPTION OF DRAWINGS

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates an exemplary embodiment of the photobiomodulation apparatus; and

FIGS. 2a and 2b illustrate an exemplary embodiment of the photobiomodulation apparatus utilizing thumb indents as ergonomic features; and

FIG. 3 illustrates an exemplary embodiment of the mouthpiece sub assembly of the photobiomodulation apparatus; and

FIG. 4 illustrates an exemplary embodiment of the mouthpiece sub assembly for targeting the base of tongue.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION OF THE INVENTION

Before describing in detail embodiments that are in accordance with the present invention, it should be observed that the embodiments reside primarily in combinations of method steps and apparatus components related to a photobiomodulation apparatus for treating oral mucositis. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

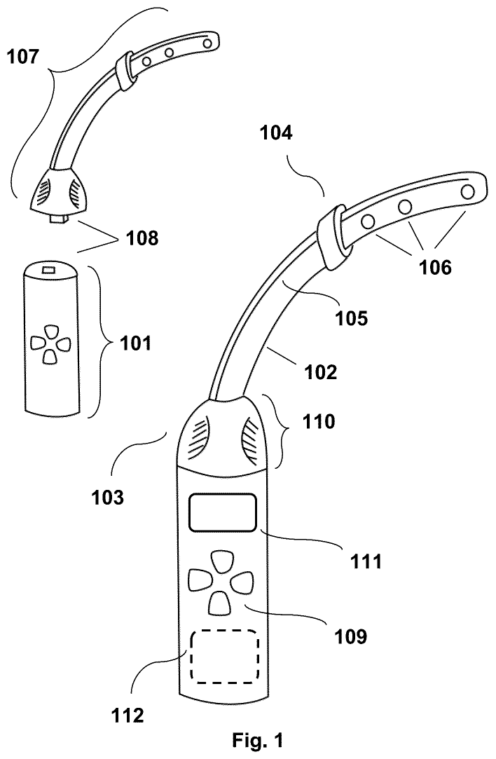

An exemplary embodiment of the photobiomodulation apparatus is shown in FIG. 1. The Photobiomodulation apparatus comprises of a handheld housing 101 and a mouthpiece subassembly 107 which are connected through an electromechanical connection 108. There is a plurality of user inputs 109, and a user feedback component 111, on the handheld housing 101. The mouthpiece subassembly 107 is comprised of a mouthpiece sub assembly base 110 connected to a transparent flexible mouthpiece housing 102 with a plurality of embedded light sources 106. A semi rigid support member 105 is encased and insulated within the transparent flexible mouthpiece housing 102. There is a single or a plurality of ergonomic features 103 either on the handheld housing 101 and/or on the mouthpiece sub assembly 107. A rechargeable battery 112 is housed in the handheld housing 101. A calibration clip 104 can be fixed on the outside of the transparent flexible mouthpiece housing 102.

The electromechanical connection 108 can either be a permanent, or a quick disconnect connection embodiment. The electromechanical connection 108 exemplified in FIG. 1 is a quick disconnect connection, meaning the user can disconnect the handheld housing 101 and the mouthpiece subassembly 107 without the aid of a separate tool. One example of a quick disconnect connection utilizes a USC type C pair of components.

The handheld housing 101 weights approximately 100 grams.

The user inputs 109 may comprise one or more of the following: buttons, switches, and voice control. The user inputs 109 allow the user to power the device on, change the parameters of the light source settings, and start and stop treatments.

The user feedback component 111 may comprise of both visual and audio indicators such as: OLED display, LCD display, LED indicators, buzzer, speakers. The user feedback component 111 assists the user by providing them with information such as: battery life indicators, LED emission status, treatment timer, mode status, etc.

The mouthpiece subassembly 107 weights approximately 20 grams.

The transparent flexible mouthpiece housing 102 is optically transparent at visible and near infrared wavelengths, comprised of a biocompatible material (for example Polyvinyl Chloride or Thermoplastic Polyurethane), with a durometer between 30 Shore A and 80 Shore D. The transparent flexible mouthpiece housing 102 is thin (less than 10 mm) and long (between 90 mm and 150 mm), which allows the apparatus to be inserted into the patient's oral cavity in a way which (1) reaches the back of the oral cavity, including the soft palate and base of tongue, and (2) can be inserted without the patient opening their mouth wide (>12 mm).

The light sources 106 preferably consist of lasers such as laser diodes, diode pumped solid state lasers (DPSSLs), or light emitting diodes (LEDs), organic light emitting diode (OLED) sheets, and electrical luminance (EL) emitters, which produce therapeutic light in a desired wavelength range. In one embodiment of the light source, a plurality of LEDs (manufacturer: OSRAM, model: GH CSSRML.24) with output power of 520 mW and output wavelength of 660 nm are used.

The light sources 106 deliver therapeutic light through the transparent flexible mouthpiece housing 102 of the apparatus to the oral mucosa and produces a variety of photochemical processes, e.g. increase of ATP (adenosine triphosphate), triggering of photo neurological response and activation of enzymes, changes in local pressure, increases in temperature, and deformation of cellular membranes. Such photochemical reactions help to reduce the incidence and severity of oral mucositis in cancer patients and help improve outcomes of cancer therapy. The light sources 106 may also comprise lamp light sources or other types of broadband light sources such as supercontinuum light sources which are integrated with optical filters to select a desired output wavelength range.

The plurality of light sources 106 can be oriented in various directions, either axially, laterally, or a combination of both. One such embodiment, as shown in FIG. 1, utilizes two light sources oriented laterally through the front face of the transparent flexible mouthpiece housing 102 and one light source oriented axially through the tip of the transparent flexible mouthpiece housing 102.

Depending on the output wavelength of the light source 106, the therapeutic light can provide a variety of treatment effects. The ultraviolet (UV)-blue light, at a wavelength of 370 to 490 nm, has high photon energy, which can help to produce singlet oxygen and effectively destroy bacteria. The red light, at a wavelength of 620 to 700 nm, can penetrate human tissue to a depth of about 8-10 mm. Skin layers, because of their high blood and water content, easily absorb red light, which helps to increase blood circulation and decrease inflammation/irritation. The near-infrared (NIR) light, at a wavelength of >700 nm, has been demonstrated to be beneficial for increasing cytochrome oxidase activity and ATP (adenosine triphosphate) content as well as promoting wound healing and relieving pain. The light sources 106 may comprise multiple light sources with different output wavelengths, each wavelength matching with the absorption band of a specific chromophore (water, hemoglobin, lipid, protein, etc.) of the subject oral mucosa.

The semi rigid support member 105 can be either one semi rigid part or a plurality of semi rigid connections between other electronic components (i.e., light sources, sensors, etc.) within the transparent flexible mouthpiece housing 102. The semi rigid support member is comprised of a malleable part, including but not limited to a malleable metal wire, or a malleable thermoplastic component. One embodiment of the semi rigid support member is a 19 Guage stainless steel wire. When the transparent flexible mouthpiece housing 102 is under force (e.g. a bending force or a moment force) by a user of the apparatus, the transparent flexible mouthpiece housing will bend. When the force is removed from transparent flexible mouthpiece housing 102, the semi rigid support member 105 will keep the transparent flexible mouthpiece housing bent at the “under force” shape.

When the transparent flexible mouthpiece housing 102 is bent, and the semi rigid support member 105 is maintaining the bent shape, a user can insert the mouthpiece subassembly 107 into their mouth thereby delivering therapeutic light from the light sources 106 in a minimally obtrusive method. This minimally obtrusive method is achieved because the transparent flexible mouthpiece housing 102 is thin, therefore the users mouth needs to be open only less than 12 mm wide. Moreover, because the semi rigid support member 105 maintains the bend shape of the transparent flexible mouthpiece housing 102, the transparent flexible mouthpiece housing can be bent into complex shapes adopting to the geometry of the patient's oral cavity, which minimize contact with the user's oral tissue when inserted into the mouth.

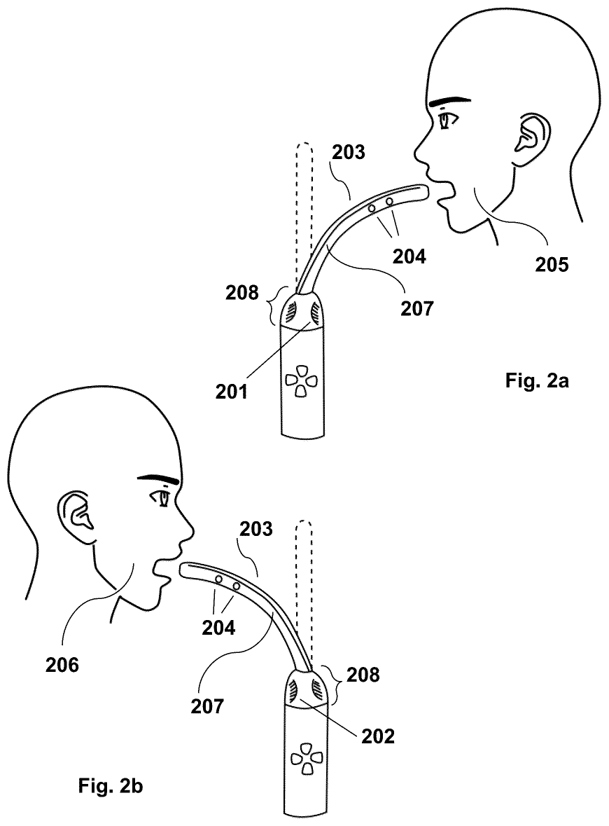

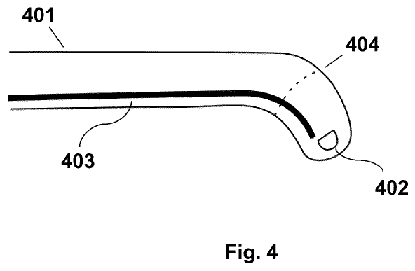

The ergonomic features 103 are a combination of mechanical and/or visual features in the handheld housing 101 and the mouthpiece sub assembly base 110 (including the transparent flexible mouthpiece housing 102). Mechanical features include, but are not limited to, embossed features, debossed features, tactile finishes, finger indentations, attached mechanical parts, and over molded parts. Visual features include, but are not limited to, pad printed and silk-screened symbols. These mechanical and visual features assist the user in bending the transparent flexible mouthpiece housing 102 into the various bend orientations which, when inserted into the patient's mouth, limit contact with the damaged oral tissue while also delivering therapeutic light to the desired target tissue. Two exemplary embodiments of the ergonomic features 103 are shown in FIG. 2 and FIG. 4.

The rechargeable battery 112 can be a Lead-Acid, Nickle-Cadmium (NiCd), Nickel-Metal Hydride (NiMH), or Lithium-Ion (Li-Ion) battery. The rechargeable battery can be accessed and replaced by removing a cover in the handheld housing 101. One embodiment of the rechargeable battery is an 18650 3.7V 2200 mAh Li-Ion battery which is changed (5V dc, 2A) via a USB-C port on the handheld housing 101.

An optional calibration clip 104 can be attached to the transparent flexible mouthpiece housing 102. The calibration clip 104 is affixed at a customizable axial position from the tip of the housing. This axial distance is carefully adjusted, by a healthcare practitioner, based on the individual patient's oral cavity geometry. The calibration clip 104 provides the patient guidance for the proper insertion depth of the apparatus. The calibration clip 104 incorporates a self-locking mechanism such as, but not limited to, a 2-part wedge, adhesive, hooks, hinges. The self-locking mechanism enables a healthcare provider to easily personalize the axial distance for each individual patient's needs.

An exemplary embodiment of the photobiomodulation apparatus with thumb indentation ergonomic features is shown in FIG. 2a and FIG. 2b. The Photobiomodulation apparatus comprises of a flexible transparent mouthpiece housing 203 connected to a mouthpiece sub assembly base 208. The mouthpiece sub assembly base 208 contains two ergonomic features: a right thumb indentation 201 and a left thumb indentation 202. A plurality of light sources 204 deliver therapeutic light through the front face of the transparent flexible mouthpiece housing 203. A semi-rigid support member 207 is embedded in the transparent flexible mouthpiece housing 203.

FIG. 2a exemplifies the bend orientation when a user (using the right thumb indentation 201 as a fulcrum) bends the transparent flexible mouthpiece housing 203 to the right. The semi-rigid support member 207 keeps the transparent flexible mouthpiece housing 203 bent after the user removes the bend force. When the user inserts the bent transparent flexible mouthpiece housing 203 into their mouth, the light sources 204 deliver therapeutic light to the patient's left side oral cavity 205. The anatomy treated in this orientation is the left buccal mucosa and the right lateral tongue border.

FIG. 2b exemplifies the bend orientation when a user (using the left thumb indentation 202 as a fulcrum) bends the transparent flexible mouthpiece housing 203 to the left. The semi-rigid support member 207 keeps the transparent flexible mouthpiece housing 203 bent after the user removes the bend force. When the user inserts the bent transparent flexible mouthpiece housing 203 into their mouth, the light sources 204 deliver therapeutic light to the patient's right side oral cavity 206. The anatomy treated in this orientation is the right buccal mucosa and the left lateral tongue border.

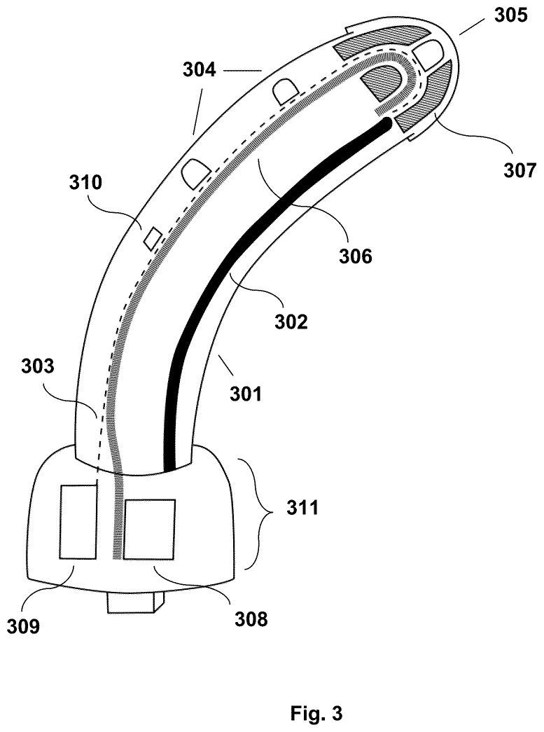

An exemplary embodiment of the mouthpiece sub assembly of the photobiomodulation apparatus is shown in FIG. 3. The mouthpiece sub assembly is comprised of a transparent flexible mouthpiece housing 301, which is connected to a mouthpiece sub assembly base 311. A semi-rigid support member 302 is electrically insulated within the transparent flexible mouthpiece housing. Enclosed within the transparent flexible mouthpiece housing 301 is a flexible electronic substrate 303, on which a plurality of light sources 304/305 is mounted (this exemplary embodiment contains two side emitting light sources 304 and a tip emitting light source 305). A flexible heat transfer substrate 306 is thermally connected to the flexible electronic substrate 303. A mounting component 307 connects the light sources 304/305, and the flexible electronic substrate 303 to the transparent flexible mouthpiece housing 301. The mouthpiece sub assembly base 311 contains a rigid electronic component 309 which is connected to the flexible electronic substrate 303. In one embodiment, a heat management solution 308 is housed within the mouthpiece sub assemble base 311 and thermally connected to the flexible heat transfer substrate 306. In another embodiment, a plurality of analog sensors 310 is included on the flexible electronic substrate 303, rigid electronic component 309, and/or the transparent flexible mouthpiece housing 301.

The transparent flexible mouthpiece housing 301 is either comprised of one or two parts. In a one-part embodiment, the distal end of the housing is sealed via induction heating, radio frequency tip forming, or other tip forming techniques. In an additional one-part embodiment, the transparent flexible mouthpiece housing 301 is created by over molding a transparent thermoplastic over the other components in FIG. 3. In a two-part embodiment, a cap is attached to the distal end of the housing and sealed by epoxy, ultrasonic welding, or other bonding methods.

The flexible electronic substrate 303 is electrically insulated within the transparent flexible mouthpiece housing 301 and is connected to the rigid electronic component 309. The flexible electronic substrate 303 is comprised of a thin passive substrate (e.g. plastic, textile, etc.) and an active electronics layer. One embodiment of the flexible electronic substrate 303 is a flexible printed circuit board. An additional embodiment of the flexible electronic substrate 303 is over molded plastic with an embedded active electronics layer or embedded components.

The flexible heat transfer substrate 306 is thermally connected to, or embedded within, the flexible electronic substrate 303. In one embodiment, the flexible heat transfer substrate 306 is embedded within the flexible electronic substrate 203, such as the copper ground layer of a flexible printed circuit board. In another embodiment, the flexible heat transfer substrate 306 is the semi-rigid support member 302 itself (for example, a 19 Guage copper wire). In another embodiment, the flexible heat transfer substrate 306 is a heat transfer fluid (i.e., water, or other coolant) filled within the transparent flexible mouthpiece housing. This fluid is cycled through the transparent flexible mouthpiece housing 301, creating a heat transfer chamber.

The mounting component 307 is specially designed with surface geometry (i.e, the radius of the surfaces around which the flexible substrates 303 and 306 are wrapped are sufficiently large) to prevent exceeding the minimum bend radius of the flexible electronic substrate 303 and the flexible heat transfer substrate 306. The mounting component 307 aligns the orientation of the light sources 304/305 relative to the transparent flexible mouthpiece housing 301. In one embodiment the flexible electronic substrate 303 is threaded inside of the mounting component 307, which is attached to the transparent flexible mouthpiece housing 301 via epoxy.

The rigid electronic component 309 is electrically connected to the flexible electronics substrate 306. In one embodiment, the rigid electronics component 309 and the flexible electronics substrate 306 are the same component (i.e. a hybrid flex-rigid printed circuit board).

The heat management component 308 is thermally connected to the flexible heat transfer substrate 306. In one embodiment, the heat management component 308 is a passive heat sink (i.e., copper, aluminum, phase change liquid, etc.) In another embodiment, the heat management component is an active heat sink (i.e., forced air, fluid loop, thermoelectric cooler, etc.)

The sensors 310, including but not limited to: thermistor, thermocouple, flex sensor, photoresistor, are included on the flexible electronic substrate 303, rigid electronic component 309, and/or the transparent flexible mouthpiece housing 301. In one embodiment, a thermistor is included on the flexible electronic substrate 303 and is used to monitor the surface temperate of the light sources 304. In another embodiment, a flex sensor is embedded within the transparent flexible mouthpiece housing 301 to measure its bend orientation. In another embodiment, a thermocouple or thermistor is imbedded near the surface of the transparent flexible mouthpiece housing 301 to measure tissue temperate.

The light sources 304/305 may be pulsed to enhance its treatment effect. The output power, duration, and pulsation property (i.e. pulse width, repetition rate, duty cycle) of the light sources 304/305 is controlled by the firmware (not shown) via the flexible electronic substrate 303 or wireless connection to adjust the light dosage applied to the oral mucosa. Such properties of light sources 304/305 may be optimized in accordance to the skin color, skin thickness, diagnosis, type of oncological intervention, and age of the patient to produce the best therapeutic effect. The firmware can toggle between the various light source orientations to emit light in different combinations and directions. The firmware can also modulate emission to allow tissue temperature to regulate (off time between 10-500 ms every 1000-6000 ms). The firmware can store the usage history of the device, which is helpful to track compliance of the self-administered treatments.

An exemplary embodiment of the photobiomodulation apparatus with an ergonomic feature near the distal end of the transparent flexible mouthpiece housing is shown in FIG. 4. The Photobiomodulation apparatus comprises of a flexible transparent mouthpiece housing 401 with an embedded light source 402 at the distal end, and a semi-rigid support member 403 embedded through its length. An ergonomic feature is on the outside surface of the flexible transparent mouthpiece housing 401 near the distal end. One embodiment of the ergonomic feature 404 is a visual feature (i.e. a silk screened or pad printed line) near the distal end of the transparent flexible mouthpiece housing 401.

FIG. 4 exemplifies the bend orientation when a user bends the transparent flexible mouthpiece housing 401 (at the ergonomic feature 404). The semi-rigid support member 403 keeps the transparent flexible mouthpiece housing 401 bent after the user removes the bend force. When the user inserts the bent transparent flexible mouthpiece housing 401 into their mouth, the light source 402 delivers therapeutic light to the patient's base of tongue an oropharynx.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

1. A photobiomodulation apparatus for providing photobiomodulation therapy to oral mucosa tissue of a patient, the photobiomodulation apparatus comprising:
a handheld housing;
a flexible mouthpiece housing being partially or fully inserted into oral cavity of the patient, wherein the flexible mouthpiece housing is attached to the handheld housing;
a semi-rigid support member encased and insulated in the flexible mouthpiece housing;
a flexible electronic substrate encased and insulated in the flexible mouthpiece housing;
at least one light source attached to the flexible electronic substrate;
wherein the flexible mouthpiece housing, the semi-rigid support member, and the flexible electronic substrate are malleable to a desired shape and orientation to (1) maintain a predetermined distance between the flexible mouthpiece housing and the targeted oral mucosa tissue of the patient such that the at least one light source emit therapeutic light through the flexible mouthpiece housing to provide photobiomodulation therapy to the targeted oral mucosa tissue, and (2) to provide a comfortable treatment by minimizing the contact with patients oral mucosa.

2. The photobiomodulation apparatus of claim 1, wherein the desired shape and orientation are maintained by the semi-rigid support member.

3. The photobiomodulation apparatus of claim 1, further comprises a plurality of ergonomic features for controlling insertion depth, shape, and orientation of the flexible mouthpiece housing in the oral cavity of the patient.

4. The photobiomodulation apparatus of claim 3, wherein the plurality of ergonomic features comprise a calibration clip affixed at a customizable axial position from the tip of the flexible mouthpiece housing for controlling insertion depth of the transparent flexible mouthpiece in the oral cavity of the patient.

5. The photobiomodulation apparatus of claim 3, wherein the plurality of ergonomic features comprises thumb indentations on the handheld housing for assisting bending of the flexible mouthpiece housing to the desired shape and orientation.

6. The photobiomodulation apparatus of claim 3, wherein the plurality of ergonomic features comprises handheld housing and/or the flexible mouthpiece housing, including but are not limited to, embossed features, debossed features, tactile finishes, finger indentations, attached mechanical parts, over molded parts, pad printed symbols, and silk-screened symbols.

7. The photobiomodulation apparatus of claim 1, further comprises a flexible heat transfer substrate thermally connected to the flexible electronic substrate to provide heat dissipation for the at least one light source.

8. The photobiomodulation apparatus of claim 1, further comprises a substrate mounting component, wherein the substrate mounting component is attached to the flexible electronic substrate and the flexible mouthpiece housing for the correct alignment of the light sources and to prevent the flexible electronic substrate from exceeding the minimum bend radius.

9. The photobiomodulation apparatus of claim 1, wherein the flexible mouthpiece housing comprised of a biocompatible material which is: at least partially transparent to the therapeutic light, has a durometer between 30 Shore A and 80 Shore D, is less than 10 mm in diameter, and between 90 mm and 150 mm long.

10. A Method for using a self-administered photobiomodulation apparatus for providing photobiomodulation therapy to oral mucosa tissue of a patient comprising:
bending a flexible mouthpiece housing using ergonomic features to guide the bending,
inserting the flexible mouthpiece housing into the mouth such that the light sources are directed towards the target oral mucosa (i.e. subsite),
turn the device into emission mode to provide therapeutic photobiomodulation light to the target oral mucosa,
keep the flexible mouthpiece housing at the subsite until the device stops emitting.

11. The method of claim 10 further comprising repeating the therapy for numerous subsites including the buccal mucosa, lateral tongue border, soft palate, ventral tongue, lips, etc.

12. The method of claim 10 further comprising a subsite timer which provides an 8 sec therapy session.

13. A method for providing photobiomodulation therapy to oral mucosa tissue of a patient, the method comprising the steps of:
providing a photobiomodulation apparatus with a flexible mouthpiece housing, the flexible mouthpiece housing comprising a flexible electronic substrate mounted on a semi-rigid support member and at least one light source attached to the flexible electronic substrate, wherein the at least one light source, the flexible electronic substrate and the semi-rigid support member are encased and insulated in the flexible mouthpiece housing;
setting an insertion depth, shape, and orientation of the flexible mouthpiece housing (by utilizing ergonomic features on the device) by a healthcare provider in accordance with the geometry of the patient's oral cavity such that the flexible mouthpiece housing maintains a predetermined distance to the targeted oral mucosa tissue of the patient to minimize contact with the patient's oral mucosa, wherein the shape and orientation of the flexible mouthpiece is maintained by the semi-rigid support member; and
inserting the flexible mouthpiece housing at the set insertion depth, shape, and orientation into the patient's oral cavity and powering on the at least one light source to emit therapeutic light through the flexible mouthpiece housing to provide photobiomodulation therapy to the targeted oral mucosa tissue.

14. The method of claim 13, further comprising a step of setting a power and duration of the at least one light source by the healthcare provider.

15. The method of claim 13, wherein the photobiomodulation apparatus further comprises a handheld housing and the flexible mouthpiece housing is attached to the handheld housing.
